(19) United States
(12) Patent Application Publication
(10) Pub. No.: US 2008/0300531 A1
(43) Pub. Date: Dec. 4, 2008

(54) Single infusion port apparatus and method for phacoemulsification

(76) Inventor: James Pitzer Gills, JR., Palm Harbor, FL (US)

Correspondence Address: KRAMER & AMADO, P.C., 1725 DUKE STREET, SUITE 240, ALEXANDRIA, VA, 22314, US

(21) Appl. No.: 11/806,115

(22) Filed: May 30, 2007

(51) Int. Cl. A61B 17/32

(52) U.S. Cl. 604/22

(57) ABSTRACT

A phacoemulsification apparatus having a single infusion port oriented toward the posterior of the eyeball and away from the cornea may be used in a process for removing cataracts in patients suffering from Fuch's Dystrophy. A method for using a phacoemulsification apparatus having a single infusion port oriented toward the posterior of the eyeball and away from the cornea to remove cataracts in patients suffering from Fuch's Dystrophy is also described.

### BACKGROUND

1. Field of the Invention

Various aspects of the invention relate generally to a technique for removal of cataracts. Other aspects of the invention relate generally to a phacoemulsification apparatus and procedures and methods for using such an apparatus for removing cataracts.

2. Description of Related Art

Phacoemulsification is a technique for removal of cataracts and involves use of an ultrasonically vibrated hollow needle to disassemble or fragment the cataract. The phacoemulsification procedure incorporates suction to remove fluid and cataract fragments from the eye through the hollow needle. To maintain ocular volume and pressure, fluid flows into the eye through a hollow sleeve having ports on its side. Two port phacoemulsification sleeves are generally used in cataract removal. The ports on the side of the sleeve allow fluid to go into the anterior chamber of the eye.

Use of single-port sleeves has been suggested for people with floppy iris syndrome. Intraoperative floppy iris syndrome has been observed during phacoemulsification cataract surgery in some patients treated with alpha-1 blockers, including Flomax. Intraoperative floppy iris syndrome is characterized by the combination of a flaccid iris that billows in response to intraoperative irrigation currents and intraoperative constriction of the pupil.

Cataract surgery may be further complicated by any number of conditions when performed with a conventional two-port phacoemulsification sleeve. One such complication is Fuchs' corneal endothelial dystrophy. In Fuch's corneal dystrophy, the inner cell layer of the cornea, or endothelium, deteriorates and becomes fragile. The use of two-port sleeves with cataract patients suffering from Fuch's corneal dystrophy may traumatize the cells of the endothelium and result in the cornea turning cloudy, due to the flow of fluid directed to the already compromised corneal endothelium.

### SUMMARY

In light of the present need for a method and apparatus for the treatment of cataracts in patient's suffering from Fuch's corneal dystrophy, a brief summary of various exemplary embodiments is presented. Some simplifications and omission may be made in the following summary, which is intended to highlight and introduce some aspects of the various exemplary embodiments, but not to limit its scope. Detailed descriptions of exemplary embodiments adequate to allow those of ordinary skill in the art to make and use the invention concepts will follow in later sections.

In accordance with an embodiment of the present invention, the process for removing a cataract in a patient suffering from Fuch's Dystrophy includes the steps of making an incision into an eyeball, the incision allowing access to an anterior chamber of the eyeball, and inserting a phacoemulsification apparatus into the anterior chamber of the eyeball.

The phacoemulsification apparatus includes a sleeve characterized by a proximal end, a distal end and a hollow bore running axially through the sleeve; and a phacoemulsification needle located in the hollow bore of the sleeve. The sleeve is characterized by a single infusion port located in the distal end of the sleeve, the single infusion port oriented toward the posterior of the eyeball and away from the cornea. The phacoemulsification needle may include a tip bent from the longitudinal axis of the phacoemulsification needle toward the posterior of the eyeball and away from the cornea.

During the process, the orientation of the single infusion port and the needle tip are oriented toward the posterior of the eyeball and away from the cornea. As a result of the orientation of the phacoemulsification apparatus, viscoelastic fluid is infused toward the corneal endothelium, as well as directing the force of phacoemulsification against the cataract in the posterior capsule and away from the cornea. By applying ultrasonic energy to break a lens having a cataract thereon into fragments using the phacoemulsification needle, the fragments are removed by suction from the anterior chamber of the eyeball. Reducing flow, and directing it posteriorly preserves the integrity of the fragile cornea associated with Fuch's corneal dystrophy.

In accordance with an embodiment of the present invention, a phacoemulsification apparatus for removing a cataract in a patient suffering from Fuch's Dystrophy includes a sleeve, directed posteriorly, characterized by a proximal end, a distal end and a hollow bore running axially through the sleeve and a phacoemulsification needle located in the hollow bore of the sleeve.

The sleeve is characterized by a single infusion port located in the distal end of the sleeve, the single infusion port oriented toward the posterior of the eyeball and away from the cornea. The phacoemulsification needle may be comprised of a tip bent between 0-30° from the longitudinal access of the needle toward the posterior of the eyeball and away from the cornea. The apparatus further includes means for maintaining the orientation of the single infusion port and the bent tip toward the posterior of the eyeball and away from the cornea.

### BRIEF DESCRIPTION OF THE DRAWINGS

In order to better understand various exemplary embodiments, reference is made to the accompanying drawings, wherein:

FIG. 1 shows a cross-sectional view of a phacoemulsification apparatus.

FIG. 2 shows a cross-section view of a distal end of a phacoemulsification apparatus

FIG. 3 shows a cross-section view of a phacoemulsification apparatus with a fixation means.

FIG. 4 shows the outside of a phacoemulsification apparatus with visual orientation indicators.

### DETAILED DESCRIPTION

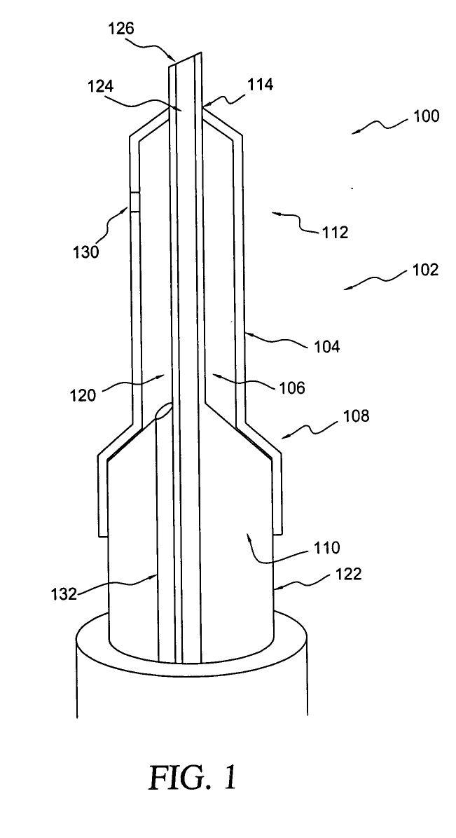

Referring now to the drawings, in which like numerals refer to like components or steps, there are disclosed broad aspects of various exemplary embodiments. FIG. 1 is a cross-sectional view of an embodiment of a phacoemulsification apparatus 100. FIG. 1 shows a sleeve 102 having a tubular wall 104 and a hollow bore running axially through wall 106. The tubular wall 104 includes an enlarged proximal end 108 having an opening 110 which is adapted to fit over a hub 122 of a phacoemulsification needle 120; and a narrow distal end 112 having an opening 114 which is adapted to fit around a tip 126 of a phacoemulsification needle 120 in a substantially water-tight manner. A single infusion port 130 is located in the tubular wall 104. The single infusion port 130 has an aperture radius r. Phacoemulsification needle 120 further includes bore 124 which exits the sleeve 102 at the distal end 112 as needle tip 126.

When the sleeve 102 of FIG. 1 is placed over a phacoemulsification needle 120 and inserted into the eye, viscoelastic solution travels through a passage 132 between the needle 120 and the inner wall of tube 104, entering from a fluid source through a passage 132 through hub 122. The viscoelastic solution is infused into the eye through the single infusion port 130. Meanwhile, lens fragments are suctioned out of the eye through a bore 124 in needle 120. The single infusion port 130 is oriented toward the posterior of the eyeball and away from the cornea. Further, the tip 126 of the needle may be oriented toward the posterior of the eyeball and away from the cornea.

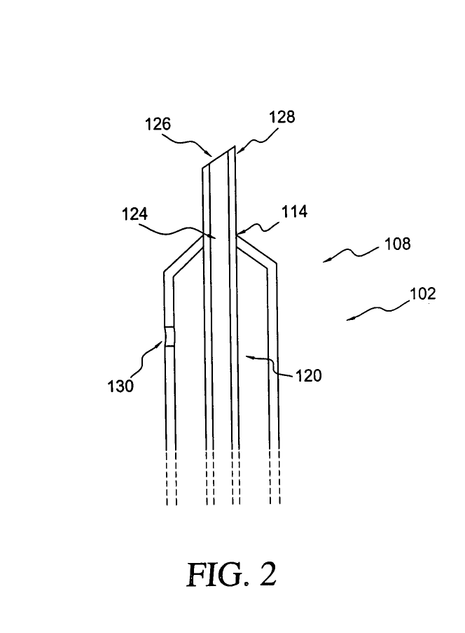

FIG. 2 is a cross-sectional view of the distal end 112 of the sleeve 102. The distal end 112 is defined as the portion of the sleeve 102 that enters the eyeball during phacoemulsification. The distal end 112 is further defined as including the single infusion port 130 and the opening 114 from which the tip 126 of the phacoemulsification needle 120 emerges from a water-tight seal. In an alternative embodiment of the tip 126, the tip 126 may include a bend 128. Bend 128 may be angled from the longitudinal axis of the needle at an angle of 0-30°. In an alternative embodiment, bend 128 may orient tip 126 toward the single infusion port 130. When in use, the single infusion port 130 and the tip 126 are oriented toward the posterior of the eyeball and away from the cornea.

The single infusion port 130 may be an aperture in the shape of a circle or an oval. Further the single infusion port 130 may include an enlarged aperture as compared to prior art infusion apertures found in two-port phacoemulsification sleeves. The enlarged size of the single infusion port may be larger than one aperture of a two-port sleeve or may be larger than the total area of both apertures of a two-port sleeve. In an embodiment the enlarged aperture of the single infusion port may be characterized by a diameter of 0.3-2.0 mm. In another embodiment, the enlarged aperture of the single infusion port may be characterized by a diameter of 1.0-2.0 mm.

Use of a one port sleeve with a larger aperture on a phacoemulsifier needle allows phacoemulsification of patients without requiring a reduction in flow rate of viscoelastic fluid or the extent of vacuum applied to the eye. Even without changing flow rate or vacuum, phacoemulsification may be performed with less disturbance of the cornea because viscoelastic fluid flows posteriorly, away from the cornea in the eye.

The phacoemulsification apparatus 100 may further include means for maintaining the orientation of the single infusion port 130 and the tip 126 toward the posterior of the eyeball and away from the cornea. In an embodiment, the means for maintaining the orientation of the single infusion aperture 130 includes a means for fixing the orientation of sleeve 102 relative to needle 120.

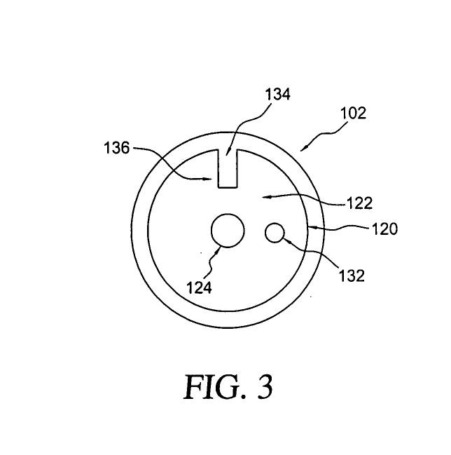

FIG. 3 shows, a sleeve 102 provided with a means for fixing the orientation of the sleeve 102 relative to the needle 120. This may take the form of a tab 134 on the inner surface of sleeve 102 which fits into a corresponding slot 136 on the hub 122 of the phacoemulsification needle 120.

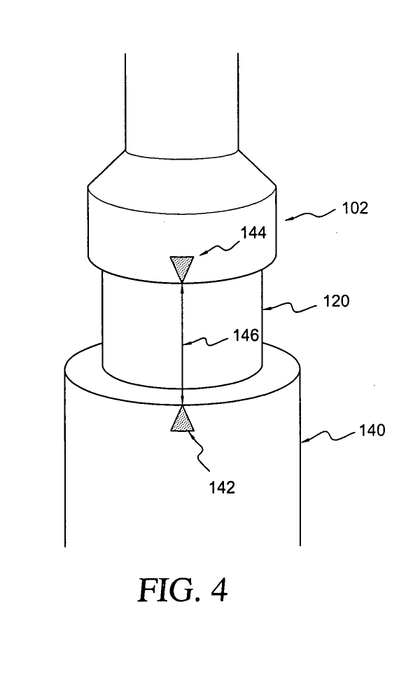

FIG. 4 shows, the proximal end 108 of the sleeve 102. The sleeve 102 is enlarged at opening 110 to fit over needle 120. Needle 120 is attached to an ultrasonic handpiece 140, which provides ultrasonic energy to the needle 120 as well as viscoelastic fluid to passage 132. The handpiece 140 may include a visual orientation indicator 142 for maintaining the orientation of the single infusion port 130 toward the posterior of the eyeball and away from the cornea. The means for maintaining the orientation of the single infusion port 130 and the tip 126 may further include a visual orientation indicator 144 located on the sleeve. The means for maintaining the orientation of the single infusion port 130 and the tip 126 may further include a visual orientation indicator 146 located on the needle. In an embodiment of the phacoemulsification apparatus 100, the visual orientation indicator 142 of the handpiece 140 may match the visual orientation indicator 144 of the sleeve 102 and the visual orientation indicator 146 of the needle 120. In an alternative embodiment, the visual orientation indicator 144 of the sleeve 102 may match the visual orientation indicator 146 of the needle 120.

The visual orientation indicators 142, 144 and 146 may include any visual indicators that can be matched to orient the various elements of the phacoemulsification apparatus. Particular examples of visual orientation indicators may include one or more of the following elements, including combinations thereof: lines, triangles, ovals, rectangles, circles, arrows, cross-hairs, letters, numbers, color coding, and partial images. In an alternative embodiment, one or more visual orientation indicators are used to orient the sleeve and needle according to a fixation means, for example a tab and slot, whereby the visual orientation indicators align the sleeve and needle according a tab and slot.

The invention further relates to a method of making an incision into an eyeball, the incision allowing access to an anterior chamber of the eyeball and inserting a phacoemulsification apparatus into the anterior chamber of the eyeball. The incision may be a scleral, a corneal or a sclerocorneal incision as long as the single infusion aperture is oriented toward the posterior of the eyeball and away from the cornea. In a preferred embodiment, the incision is a scleral incision.

The phacoemulsification apparatus includes a sleeve characterized by a proximal end, a distal end and a hollow bore running axially through the sleeve; and a phacoemulsification needle located in the hollow bore of the sleeve. The sleeve is characterized by a single infusion port located in the distal end of the sleeve, the single infusion port oriented toward the posterior of the eyeball and away from the cornea. The phacoemulsification needle may include a tip bent from the longitudinal access of the phacoemulsification needle toward the posterior of the eyeball and away from the cornea. Prior to initiating the removal of the cataract, the user of the phacoemulsification device orients the single infusion port toward the posterior of the eyeball and away from the cornea. The user may further orient the tip of the needle toward the posterior of the eyeball and away from the cornea. The orientation may be accomplished by mechanical means or visual orientation indicators located within the sleeve, the needle, and/or the handpiece.

The phacoemulsification needle applies ultrasonic energy in the form of ultrasonic vibration to break the lens of the eye, which has a cataract thereon, into fragments. These fragments, along with a portion of the aqueous humor in the anterior chamber of the eye, are removed by suction from the anterior chamber through the bore of the needle. If desired, a chopper may be inserted through a second incision to help with chopping the nucleus into smaller pieces. A viscoelastic fluid is supplied from an irrigation source to the anterior chamber of the eyeball through the single infusion port so as to coat and cover the corneal endothelium. The viscoelastic fluid supports the cornea and helps maintain the shape of the eyeball. The viscoelastic is preferably refreshed every few seconds.

The viscoelastic fluid is supplied to the anterior chamber of the eyeball through a tubular sleeve 102 shown in FIG. 1-4. The sleeve 102 includes a hollow bore 106 running axially through the sleeve 102 and a single infusion aperture 130 in the distal end 112, wherein the phacoemulsification needle 120 runs through the hollow bore 106 of the sleeve 102 so as define a space between the needle and an inner surface of the sleeve 102. Viscoelastic fluid travels from the handpiece 140, through a passage 132 running through the hub 122 of the phacoemulsification needle 120, into the sleeve 102. The viscoelastic fluid is then infused through the single infusion port 130 oriented toward the corneal endothelium. Therefore, the viscoelastic fluid is infused toward the posterior of the eyeball and away from the cornea. Lens fragments created by the application of ultrasonic energy are suctioned out of the anterior chamber of the eye through bore 124. The viscoelastic fluid in the anterior segment coats the endothelium. The coating is refreshed every several seconds of phacoemulsification.

In a preferred embodiment, the needle tip 126 is further bent in the direction of the single infusion aperture 130. The bent tip 126 therefore directs the ultrasonic energy to break the lens against the cataract in the posterior capsule and away from the cornea.

In carrying out the method described above, the needle is positioned on an ultrasonic handpiece, and the sleeve is positioned on the needle in a fixed orientation relative to the handpiece. Preferably, the sleeve is positioned on the needle so that the viscoelastic fluid exits the sleeve toward the posterior of the eyeball and away from the cornea.

Utilizing the technique of the single port phacoemulsifier with the viscoelastic the majority of corneas remain clear postoperatively. Typical cataract surgery performed on patients with Fuch's Dystrophy would normally require corneal grafts or a triple procedure. However, with the single port phacoemulsifier the turbulence is directly posteriorly, resulting in less disturbance to the cornea, and allowing it to remain clear. The use of the single port further takes the flow of fluid away from the cornea and the emphasis of the power is distributed away from the cornea toward the center of the cataract which is being phacoemulsified. This allows for cataract surgery to be done with less interference of the endothelium of the cornea and thus preserves the corneal clarity and lessens the necessity for corneal transplantation.

In an alternative embodiment the process using the single infusion port oriented toward the posterior of the eyeball and away from the cornea includes infusing the balanced salt irrigating solution at a low flow rate of about 1-22 cc/min.

In an alternative embodiment, the process using the needle tip oriented toward the posterior of the eyeball and away from the cornea includes applying ultrasonic energy at low power. Power in phacoemulsification procedures is defined by those of skill in the art as mean power percentage used during the procedure. Mean power percentage applies to both sonic and ultrasonic devices and procedures. With regards to the definition of lower power in the present invention, low power is defined as a mean power percentage of about 1-40%. In an alternative embodiment, low power is defined as a mean power percentage of about 1-20%. In another embodiment, low power is defined as a mean power percentage of about 1-10%.

The methods described above may be used in patients with Fuch's Dystrophy. Fuch's Dystrophy is characterized by corneal swelling and clouding as a result of deterioration of the endothelial cells on the back of cornea. The method of removing a cataract using the single infusion port phacoemulsifier, oriented toward the posterior of the eyeball and away from the cornea, allows the removal of cataracts from patients with Fuch's Dystrophy, particular patients having a corneal thickness of up to about 700 microns, wherein further trauma to the weakened cornea is alleviated.

Although the various exemplary embodiments have been described in detail with particular reference to certain exemplary aspects thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing abstract, disclosure, description, and figures are for illustrative purposes only, and do not in any way limit the invention, which is defined only by the claims.

What is claimed is:

1) A process for removing a cataract in a patient suffering from Fuch's Dystrophy comprising:
making a incision into an eyeball, the incision allowing access to an anterior chamber of the eyeball;
inserting a phacoemulsification apparatus into the anterior chamber of the eyeball;
the phacoemulsification apparatus comprising:
a sleeve characterized by a proximal end, a distal end and a hollow bore running axially through the sleeve; and
a phacoemulsification needle located in the hollow bore of the sleeve;
wherein the sleeve consists of a single infusion port located in the distal end of the sleeve, the single infusion port oriented toward the posterior of the eyeball and away from the cornea;
wherein the phacoemulsification needle comprises a tip bent 0-30° from the longitudinal access of the phacoemulsification needle toward the posterior of the eyeball and away from the cornea,
maintaining the orientation of the single infusion port and the tip toward the posterior of the eyeball and away from the cornea;
infusing viscoelastic fluid from the single infusion port toward the posterior of the eyeball and away from the cornea;
directing the force of phacoemulsification against the cataract in the posterior capsule and away from the cornea;
applying ultrasonic energy to break a lens having a cataract thereon into fragments using the phacoemulsification needle;
removing the fragments, along with a portion of aqueous humor, by suction from the anterior chamber of the eyeball;
wherein the process preserves the integrity of the fragile cornea associated with Fuch's corneal dystrophy.

2) The method of claim 1, wherein the orientation of the single infusion port and the tip are maintained by positioning the sleeve on an ultrasonic handpiece, where the sleeve is positioned on the needle in a fixed orientation relative to the handpiece.

3) The method of claim 2, wherein the sleeve is positioned in a fixed orientation by a tab on an internal surface of the sleeve, and a tab-receiving slot on a hub of the needle.

4) The method of claim 1, wherein the orientation of the single infusion port and the tip are maintained by positioning the sleeve on an ultrasonic handpiece, where the sleeve and tip are oriented manually through the use of a visual orientation indicator located on the handpiece.

5) The method of claim 4, wherein the orientation of the single infusion port and the tip are maintained by positioning a visual orientation indicator located on the sleeve.

6) The method of claim 5, wherein the orientation of the single infusion port and the tip are maintained by orienting a visual orientation indicator on the needle to match with the previously positioned visual orientation indicator located on the sleeve.

7) The method of claim 1, wherein the orientation of the single infusion port and the tip are maintained by directing the bent tip toward the single infusion port.

8) The method of claim 1, wherein the single infusion port is characterized by a diameter in the range of about 1.0-2.0 mm.

9) The method of claim 1, wherein the viscoelastic fluid is infused at a low flow rate in the range of about 1-22 cc/min.

10) The method of claim 1, wherein the ultrasonic energy is applied at low power in the range of about 1-20% mean power percentage.

11) The method of claim 1 wherein the fragile cornea is characterized by a corneal thickness of equal to or less than about 700 microns.

12) A phacoemulsification apparatus for removing a cataract in a patient suffering from Fuch's Dystrophy comprising:
a sleeve characterized by a proximal end, a distal end and a hollow bore running axially through the sleeve;
wherein the sleeve consists of a single infusion port located in the distal end of the sleeve, the single infusion port oriented toward the posterior of the eyeball and away from the cornea;
a phacoemulsification needle located in the hollow bore of the sleeve;
wherein the phacoemulsification needle comprises a tip bent between 0-30° degrees from the longitudinal access of the phacoemulsification needle toward the posterior of the eyeball and away from the cornea; and
means for maintaining the orientation of the single infusion port and the bent tip toward the posterior of the eyeball and away from the cornea.

13) The apparatus of claim 12, wherein the means for maintaining the orientation of the single infusion port and the tip further comprises an ultrasonic handpiece for mounting the sleeve, where the sleeve is positioned on the needle in a fixed orientation relative to the handpiece.

14) The apparatus of claim 13, wherein means for maintaining the orientation of the single infusion aperture and the tip further comprises a tab on an internal surface of the sleeve, and a tab-receiving slot on a hub of the needle.

15) The apparatus of claim 12, wherein the fixed orientation of the tip fixes the 1-30° angle of the tip toward the single infusion aperture.

16) The apparatus of claim 12, wherein the means for maintaining the orientation of the single infusion port and the tip further comprises a first visual orientation indicator located on the handpiece, where the sleeve and tip are oriented manually through the use of the visual orientation indicator located on the handpiece.

17) The apparatus of claim 16, wherein the means for maintaining the orientation of the single infusion port and the tip further comprises a second visual orientation indicator located on the sleeve.

18) The apparatus of claim 17, wherein the means for maintaining the orientation of the single infusion port and the tip further comprises a third visual orientation indicator located on the needle, wherein the visual orientation locator of the needle matches the visual orientation indicator of the sleeve.

19) The apparatus of claim 12, wherein the single infusion port is characterized by a diameter in the range of about 1.0-2.0 mm.
